(19) United States
(12) Patent Application Publication
(10) Pub. No.: US 2007/0043030 A1
(43) Pub. Date: Feb. 22, 2007

(54) Pharmaceutical compositions for treating premature ejaculation by pulmonary inhalation

(75) Inventors: David Morton, Wiltshire (GB); John Staniforth, Wiltshire (GB); Mike Tobyn, Wiltshire (GB); Stephen Eason, Wiltshire (GB); Quentin Harmer, Wiltshire (GB); David Ganderton, Wiltshire (GB)

Correspondence Address: DAVIDSON, DAVIDSON & KAPPEL, LLC, 485 SEVENTH AVENUE, 14TH FLOOR, NEW YORK, NY, 10018, US

(73) Assignee: Vectura Limited, Chippenham, Wiltshire (GB)

(21) Appl. No.: 10/570,937

(22) PCT Filed: Sep. 15, 2004
(86) PCT No.: PCT/GB04/03935

§ 371 Date: Jul. 17, 2006

(30) Foreign Application Priority Data
Sep. 15, 2003 (GB) 0321612.4
Jun. 4, 2004 (GB) 0412562.1

(51) Int. Cl. A61K 31/5513; A61K 31/5415

(52) U.S. Cl. 514/221; 514/225.8

(57) ABSTRACT

The present invention relates to improved formulations for the treatment of premature ejaculation and, in particular, relates to the administration of antidepressants by pulmonary inhalation for treating premature ejaculation. Various types of known antidepressants may be used, including tricyclic antidepressants, such as clomipramine.

### DESCRIPTION

The present invention relates to improved formulations for the treatment of premature ejaculation and, in particular, relates to the administration of antidepressants by pulmonary inhalation for treating premature ejaculation. Various types of known antidepressants may be used, including tricyclic antidepressants, such as clomipramine.

Premature ejaculation (PE) is the persistent or recurrent ejaculation with minimal stimulation before, on or shortly after penetration and before the patient (or partner) wishes it. An occasional instance of PE might not be cause for concern, but if the problem occurs more frequently, a dysfunctional pattern usually exists for which treatment may be appropriate.

Male sexual stimulation can be classified according to functional activities during the sexual cycle. The normal male sexual response cycle is divided into five interrelated events that occur in a defined sequence: libido, erection, ejaculation, orgasm and detumescence.

Ejaculation is controlled by sympathetic innervation of the genitals and occurs as a result of a spinal cord reflex, although there is also considerable voluntary inhibitory control. Ejaculation involves two processes. Emission is associated with the secretion of seminal fluid into the posterior urethra via contractions of the ampulla of the vas deferens, seminal vesicles and prostate smooth muscle. This is followed by the second phase of expulsion of the seminal fluid through the penis to the outside. An inhibitory effect on ejaculation is thought to be mediated via serotonergic neurotransmission in the forebrain.

In normal development, men are able to control their ejaculation by the age of 17 or 18.

A spectrum of ejaculatory disorders exists, ranging from premature ejaculation through to absence of ejaculation. Premature ejaculation is described as the most common male sexual dysfunction with an estimated prevalence of around 30%. This estimate varies between 1% and 75% depending on the population and the criteria used to define the condition.

A descriptive definition that has been used defines premature ejaculation as: “persistent or recurrent ejaculation with minimum sexual stimulation that occurs before, upon or shortly after penetration and before the person wishes it and in the absence of substance abuse”. The condition can cause great distress and can place strain on relationships. Therefore, an effective and reliable treatment of PE is highly desirable.

A quantitative definition, the Intravaginal Ejaculatory Latency Time (IELT), has also been used as an endpoint to enable the assessment of interventions designed to improve ejaculatory delay. A person is considered to have premature ejaculation if the IELT is ≦60 seconds.

Premature ejaculation can be physiological in nature (neurological abnormality, acute physical illness, physical injury or pharmacological side effect) or psychological (distress, anxiety, deficit in psychosexual skill). Primary premature ejaculation describes the condition in someone who has had symptoms from the onset of sexual experience, whereas secondary PE is a sequelae to another condition, for example erectile dysfunction.

PE may be related to a number of different factors including a hypersensitive nervous system, penile sensitivity, somatic vulnerability, lack of inhibitory effect of the serotonergic system and superior reproductive strategy.

It is believed that ejaculation delay is related to 5HT_{2C} activation, with faster ejaculation associated with 5HT_{1A} activation. It is hypothesised that low 5HT neurotransmission or hypofunction of the 5HT_{2C} receptor or hyperfunction of 5HT_{1A} leads to PE.

Treatment of premature ejaculation can be divided into either psychological and behavioural counselling or drug therapy. The former can take a number of forms but all are centred on the basic procedure of the stop-start technique. This involves the man or his partner stopping stimulation and squeezing the penis, proximal to the frenulum, at the moment immediately before ejaculation. Used in a graduated fashion starting with masturbation and ending with active intercourse this technique has high initial success (60-90%) although this may decline over the 3 years after therapy to 25%.

There are a number of different drug therapy approaches to premature ejaculation. Much of the early work was done using the tricyclic antidepressants, such as clomipramine, which acts centrally via the 5HT2 receptor to inhibit serotonin reuptake, thereby promoting serotonin activity and effecting a delay in ejaculation.

Daily oral doses of 25-50 mg of clomipramine were found to be effective in delaying rapid ejaculation in Althof, et al. (J Clin Psychiatry (September 1995) 56:9, p. 402-407). It was concluded from the results of the study that clomipramine is effective in significantly lengthening ejaculatory latencies and increasing sexual and relationship satisfaction. It was also considered to be a cost-effective chronic therapy for selected patients.

There are side effects associated with the use of clomipramine in treating PE, such as spontaneous orgasm, anorgasmia, and ejaculatory pain. Additionally, there are a range of frequently reported side effects (>10%) for the oral formulation used for antidepressive indications, including dry mouth, sweating, constipation, blurred vision, nausea, drowsiness, headache and dizziness.

Work has also been carried out with selective serotonin reuptake inhibitors (SSRIs) such as sertraline (Zoloft™), fluoxetine (Prozac™) and paroxetine (Paxil™). All of these active agents have been found to be effective in producing a delay in ejaculation following oral administration, although there is generally a significant delay between administration (by ingestion) and the onset of the therapeutic effect. At present, none of these SSRIs are approved for use in treating PE.

Some early work has been done with alpha-adrenergic receptor blockers, based on the hypothesis that the sympathetic nervous system is responsible for the control of the peristaltic movement of seminal fluid. However, no definitive dosing regimen has been established in larger trials.

Abdel-Hamid, et al. (Int J Impot Res (2001) Feburary; 13(1):41-5) conducted a randomised, double blind, crossover, comparative study in 31 male patients with primary PE. The study evaluated five different therapies (clomipramine, sertraline, paroxetine, sildenafil and the “squeeze technique”) during a 4-week treatment period with a 2-week washout period. The drugs were administered orally some 3 to 5 hours before planned intercourse and not more than twice a week. It was concluded that orally administered clomipramine, sertraline and paroxetine demonstrated comparable efficacy, with sildenafil demonstrating optimal efficacy. It was also found that the “on demand” use of the drugs was associated with mild and low incidence of side effects when compared with the continuous administration proposed by earlier studies, such as Althof, et al., discussed above.

A number of new products are also currently under development, including dapoxetine, a 5HT modulator-reuptake inhibitor, 5HT3 receptor antagonists and 5HT4 antagonist, and novel fluoxetine formulations.

Limited data are available for the use of topical anaesthetic creams applied to the glans penis and penile shaft in association with the use of a condom. This treatment has not been formally tested. It seems that analgesia is maximal 2-3 hours after application and lasts for 1-2 hours depending on method of application.

The vast majority of the drug treatments for PE discussed in the prior art involve oral administration of the active agent. Whilst this is convenient, as oral dosage forms of the antidepressants tend to be readily available, this route of administration provides a relatively slow onset of the therapeutic effect, even when the oral dosage forms are formulated for rapid release of the active agent.

All the treatments discussed briefly above rely on a high degree of predictability and planning of sexual activity because of the delay between dosing and attainment of effect. It is therefore an aim of the present invention to provide a treatment for premature ejaculation which has a rapid onset of the desired therapeutic effect with minimum but adequate duration, thereby allowing important spontaneity of sexual activity and creating a much more patient-friendly treatment than currently exists. Preferably, the onset will be almost instantaneous following administration.

In addition, the present invention also seeks to avoid the side effects frequently associated with some of the known treatments discussed above. It is envisaged that this might be achieved by more efficient administration, so that smaller doses of the therapeutic agent may be administered to achieve the same therapeutic effect. It has also been noted that the side effects associated with the administration of clomipramine, such as spontaneous orgasm, anorgasmia, and ejaculatory pain may be due to the relatively unpredictable nature of oral route metabolism and so it may be possible to avoid them by using a more predictable mode of administration.

Side effects should also be reduced if the therapeutic agent can be administered on an “as needed” basis, rather than continuously, by chronic daily dosing.

According to a first aspect of the present invention, new pharmaceutical compositions comprising an antidepressant are provided for treating premature ejaculation by pulmonary inhalation.

This mode of administration preferably leads to the avoidance of, or reduction in, side effects normally associated with the administration of the antidepressant. It is especially preferred that the compositions of the present invention have an extremely rapid onset of the therapeutic effect, thereby allowing true “on demand” administration only a very short time before sexual activity. The speed of onset of the therapeutic effect for the compositions of the present invention is discussed in greater detail below.

Antidepressants are drugs that relieve the symptoms of depression. They were first developed in the 1950s and have been used regularly since then. The so-called tricyclic antidepressants (TCAs or TCADs) and the selective serotonin reuptake inhibitors (SSRIs) probably account for about 95% of antidepressants prescribed. The selective serotonin and noradrenaline reuptake inhibitors (SNRIs) are a newer group of antidepressants, but they are not yet so widely used.

Antidepressants are used to treat moderate to severe depressive illnesses. They are also used to help the symptoms of severe anxiety, panic attacks and obsessional problems. They may also be used to help people with chronic pain, eating disorders and post-traumatic stress disorder. The mechanisms by which the various antidepressants are thought to work vary considerably between the various types of antidepressants.

There are a number of different types of antidepressant drugs and these tend to fall into the following categories:

1) tricyclic antidepressants (TCADs or TCAs), such as clomipramine, imipramine, lofepramine, nortriptyline, amitriptyline, desipramine, dosulepin, doxepin, trimipramine, amoxapine, trazodone, amineptine, dothiepin, iprindole, opipramol, propizepine, protriptyline, quinupramine and fluphenazine;

2) selective serotonin and noradrenaline reuptake inhibitors (SNRIs), such as venlafaxine and milnacipran;

3) selective serotonin reuptake inhibitors (SSRIs), such as citalopram, escitalopram, fluoxetine, fluvoxamine, paroxetine, clovoxamine, femoxetine, ifoxetine, viqualine, zimeldine and sertraline;

4) selective noradrenaline reuptake inhibitors (NARIs), such as reboxetine, desipramine, oxaprotiline and melitracen;

5) noradrenaline and selective serotonin antidepressants (NASSAs), such as sibutramine and mirtazapine;

6) monoamine oxidase inhibitors (MAOIs), such as moclobemide, tranylcypromine, brofaromine, clorgyline, isocarboxazid, nialamide, pirlindole, selegiline, toloxatone, viloxazine and phenelzine;

7) lithium salts, such as lithium carbonate and lithium citrate;

8) GABA potentiators, such as valproic acid;

9) thioxanthenes, such as flupentixol;

10) tetracyclic antidepressants, such as maprotiline, levoprotiline, mianserin; and

11) further agents which may not fit into the above mentioned categories, such as bupropion, carbamazepine, tryptophan, amesergide, benactyzine, butriptyline, cianopramine, demexiptiline, dibenzepin, dimetacrine, etoperidone, fezolamine, medifoxamine, metapramine, methylphenidate, minaprine, nomifensine, oxaflozane, oxitriptan, rolipram, setiptiline, teniloxazine, tianeptine, tofenacin and nefazodone.

The term antidepressants, as used herein, may also encompass antipsychotic drugs which may also be used in the compositions of the present invention. Such antipsychotic drugs include, for example, aripiprazole, chlorpromazine, zuclopenthixol, clozapine, flupentixol, sulpiride, perphenazine, fluphenazine, haloperidol, thioridazine, pericyazine, levomepromazine, pimozide, oxypertine, pipotiazine, promazine, risperidone, quetiapine, amisulpride, trifluoperazine, prochlorperazine, zotepine and olanzapine.

Any of the abovementioned types or classes of antidepressants (for example, tricyclic antidepressants) may be used in the present invention to treat PE. What is more, any individual antidepressant mentioned above (for example, clomipramine) may also be used to treat PE.

In one embodiment of the invention, the antidepressant included in the composition is a tricyclic antidepressant. To varying extents, all of the abovementioned tricyclic agents share the capability of inhibiting the neuronal uptake of norepinephrine. That said, these tricyclic agents may vary in the severity of their side effects, most notably in the degree of sedation and the extent of the anticholinergic effects.

Clomipramine (3-chloro-5-[3-(dimethylamino)-propyl]-10,11-dihydro-5H-dibenz[b,f]azepine) is one of the preferred active agents used in the present invention. This tricyclic agent has both antidepressant and anti-obsessional properties. Lie other tricyclic antidepressants, clomipramine inhibits norepinephrine and serotonin uptake into central nerve terminals, possibly by blocking the membrane-pump of neurons, thereby increasing the concentration of transmitter monoamines at receptor sites. Clomipramine is presumed to influence depression as well as obsessive and compulsive behaviour through its effects on serotonergic neurotransmission. The actual neurochemical mechanism is unknown, but clomipramine's capacity to inhibit serotonin reuptake is thought to be important. Clomipramine also appears also to have a mild sedative effect which may be helpful in alleviating the anxiety component often accompanying depression.

As with other tricyclic compounds, clomipramine possesses anticholinergic properties which are responsible for some of its side effects. It also has weak antihistamine and antiserotonin properties, lowers the convulsive threshold, potentiates the effect of norepinephrine and other drugs acting on the CNS, has a quinidine-like effect on the heart and may impair cardiac conduction.

Clomipramine is commercially available in the form of oral tablets or capsules, usually comprising 10, 25, 50 or 75 mg of clomipramine or clomipramine hydrochloride. Absorption of clomipramine is reported to be rapid and complete after oral administration. Plasma levels usually peak some two hours after dosage but much individual variation occurs. The plasma half-life after a single oral dose is approximately 21 hours, although the active metabolite desmethylclomipramine has a half-life life of around 36 hours following oral administration.

Whilst clomipramine has been shown to be effective in treating PE with oral doses starting from about 25 mg, the onset of the therapeutic effect of the drug is relatively slow and this does present problems and can destroy the spontaneity of sexual intercourse. Furthermore, doses of clomipramine of this magnitude are associated with a variety of side effects, most of which are mild, although some of which can be serious.

On demand use of clomipramine to treat PE has been suggested in U.S. Pat. No. 6,495,154. Although it is suggested in this patent that the drug may be administered less than 30 minutes prior to engaging in sexual activity, there is actually no evidence provided to support this claim. There is also no disclosure of a dosage form or mode of administration which is likely to reliably and reproducibly provide such a rapid onset of the therapeutic effect in all patients.

It has now been discovered that antidepressants are rapidly absorbed from the lung and provide an extremely rapid onset of their therapeutic effect. In fact, the onset of the therapeutic effect is significantly faster following pulmonary administration than that observed following oral administration of tablets and the like, even where the tablets are formulated for fast release of the active agent.

Additionally, it has been found that the amount of antidepressant required to treat sexual dysfunction when said dose is administered by pulmonary inhalation is significantly smaller than the doses provided by the currently available forms of antidepressants, which are intended for oral administration.

What is more, it has also been found that administering antidepressants by pulmonary inhalation leads to an extremely beneficial pharmacokinetic profile which provides an exceptionally fast onset of the therapeutic effect with a short but sufficient and suitable duration and subsequent fast elimination of the drug from the plasma. This is in contrast to the pharmacokinetics of the orally administered tablets which exhibit a relatively slow onset of the therapeutic effect and a long presence of the drug in the plasma, presumably due to the more gradual absorption of the drug.

Advantageously, it has also been found that the small dose of an antidepressant administered by pulmonary inhalation and the fast onset and fast offset of the effect (provided by the rapid rise in drug plasma concentration, followed by the rapid fall thereof) observed as a result leads to a reduced incidence of side effects generally associated with the administration of the drugs. Most antidepressants are associated with relatively mild side effects, such as drowsiness, dry mouth, nausea, etc. These side effects are generally thought to be dose-dependent, as well as being linked to chronic administration of the antidepressants. Thus, these side effects may be reduced or avoided altogether as a result of the pulmonary administration of the antidepressants, as provided in the present invention.

In accordance with another aspect of the present invention, new methods of treating premature ejaculation are provided, using new pharmaceutical compositions comprising an antidepressant, wherein the compositions are administered by pulmonary inhalation.

Once again, these methods preferably achieve the desired therapeutic effect quickly, by virtue of a rapid onset of the effect of the antidepressant following pulmonary administration. Furthermore, the methods preferably also avoid or involve reduced side effects that are normally or frequently associated with the administration of the antidepressant, especially when they are administered orally.

According to one embodiment of the invention, the preferred antidepressant is a tricyclic antidepressant. In another embodiment, the tricyclic antidepressant is clomipramine. The term “clomipramine” as used herein includes clomipramine and clomipramine hydrochloride, as well as any other derivatives of clomipramine. Other suitable tricyclic antidepressants include those mentioned above, such as imipramine, amiprityline and doxepin.

The compositions of the present invention may comprise two or more different antidepressants, which may be from the same class or type of antidepressant (such as two different tricyclic antidepressants) or from two or more different classes (such as one or more SSRIs and one or more MAOIs). What is more, the compositions of the present invention can also additionally comprise other therapeutic agents which may optionally assist the treatment of premature ejaculation.

The additional therapeutic agents to be included in the compositions of the present invention may be one or more of the following:

1) serotonin agonists, including 2-methyl serotonin, buspirone, ipsaperone, tiaspirone, gepirone, lysergic acid diethylamide, ergot alkaloids, 8-hydroxy-(2-N,N-dipropylamino)-tetraline, 1-(4-bromo-2,5-dimethoxyphenyl)-2-aminopropane, cisapride, sumatriptan, m-chlorophenylpiperazine, trazodone, zacopride and mezacopride;

2) serotonin antagonists, including ondansetron, granisetron, metoclopramide, tropisetron, dolasetron, trimethobenzamide, methysergide, risperidone, ketansetin, ritanserin, clozapine, amitryptiline, R(+)-α-(2,3-dimethoxyphenyl)-1-[2-(4-fluorophenyl)ethyl]-4-piperidin e-methanol azatadine, cyproheptadine, fenclonine, dexfenfluramine, fenfluramine, chlorpromazine and mianserin;

3) adrenergic agonists, including methoxamine, methpentermine, metaraminol, mitodrine, clonidine, apraclonidine, guanfacine, guanabenz, methyldopa, amphetamine, methamphetamine, epinephrine, norepinephrine, ethylnorepinephrine, phenylephrine, ephedrine, pseudoephedrine, methylphenidate, pemoline, naphazoline, tetrahydrozoline, oxymetazoline, xylometazoline, phenylpropanolamine, phenylethylamine, dopamine, dobutamine, colterol, isoproterenol, isotharine, metaproterenol, terbutaline, metaraminol, tyramine, hydroxyamphetamine, ritodrine, prenalterol, albuterol, isoetharine, pirbuterol, bitolterol fenoterol formoterol, procaterol, salmeterol, mephenterine and propylhexedrine;

4) adrenergic antagonists, including phenoxybenzamine, phentolamine, tolazoline, prazosin, terazosin, doxazosin, trimazosin, yohimbine, ergot alkaloids, labetalol, ketanserin, urapidil, alfuzosin, bunazosin, tamsulosin, chlorpromazine, haloperidol, phenothiazines, butyrophenones, propranolol, nadolol, timolol, pindolol, metoprolol, atenolol, esmolol, acebutolol, bopindolol, carteolol, oxprenolol penbutolol carvedilol, medroxalol, naftopidil, bucindolol, levobunolol, metipranolol, bisoprolol, nebivolol, betaxolol, carteolol, celiprolol sotalol, propafenone and indoramin;

5) adrenergic neurone blockers, including bethanidine, debrisoquine, guabenxan, guanadrel, guanazodine, guanethidine, guanoclor and guanoxan;

6) benzodiazepines, including alprazolam, brotizolam, chlordiazepoxide, clobazepam clonazepam, clorazepate, demoxepam, diazepam, estazolam, flurazepam, halazepam, lorazepam, midazolam, nitrazepam, nordazapam, oxazepam, prazepam, quazepam, temazepam and triazolam;

7) neuroleptics, including chlorpromazine, triflupromazine, mesoridazine, thioridazine, acetophenazine, fluphenazine HCL perphenazine, prochlorperazine, trifluoroperazine, chlorprothixene, thiothixine, haloperidol, loxapine, molindone, clozapine, risperidone, olanzapine and quetiapine;

8) alpha blockers, including prazosin, phenoxybenzamine, doxazosin, terazosin, carvadilol and labetalol;

9) anxiolytics, including chlordiazpoxide, lorazepam and alprazolam; and

10) smooth muscle relaxants, including papaverine, phentolamine, cimetropium bromide, hyoscine butyl bromide, mebeverine, otilium bromide, pinaverium bromide, trimebutine and combinations thereof.

Particularly preferred additional active agents include benzodiazepines, such as those listed above.

The compositions and methods of the present invention provide a fast onset of the desired therapeutic effect. In particular, the onset is significantly faster than that observed upon oral administration of antidepressants. In one embodiment of the invention, the onset of the therapeutic effect delaying ejaculation is less than 30 minutes from the administration of the composition via the pulmonary route. In other embodiments, the time from administration to onset of the therapeutic effect is no more than 25 minutes, no more than 20 minutes, no more than 15 minutes, no more than 10 minutes, no more than 8 minutes, no more than 6 minutes, no more than 5, 4, 3 or 2 minutes, or even no more than 1 minute.

The delay to onset of the therapeutic effect following pulmonary administration of the compositions of the present invention are significantly faster than the delays disclosed in the prior art, even where the prior art has referred to “rapid onset” and “on demand” administration.

It is considered that, given the nature of the condition to be treated in the present invention, treatment cannot truly be said to be “on demand” unless the therapeutic effect provided by the composition is achieved within a period of less than 30 minutes, and really no more than 20 minutes. This is because maintaining the spontaneity of sexual intercourse plays a very important role in the treatment of PE, at the very least psychologically. Indeed, maintaining this spontaneity can even further assist the treatment of PE, beyond the effect of the antidepressant.

The present invention also relates to high performance inhaled delivery of antidepressants, which has a number of significant and unexpected advantages over oral administration. These advantages are discussed in greater detail below. It is the mode of administration and the formulations of the present invention that make this excellent performance possible.

In accordance with one embodiment of the present invention, the pharmaceutical composition is in the form of a dry powder. Preferably, the dry powder is dispensed using a dry powder inhaler (DPI).

In one embodiment of the present invention, the composition comprises active particles comprising an antidepressant, the active particles having a mass median aerodynamic diameter (MMAD) of no more than about 10 μm.

In another embodiment of the present invention, the composition comprises active particles comprising an antidepressant and an additive material which is an anti-adherent material and reduces cohesion between the particles in the composition.

In yet another embodiment of the present invention, the composition comprises active particles comprising an antidepressant and carrier particles of an inert excipient material, such as lactose. The carrier particles may have an average particle size of from about 5 to about 1000 μm.

In an alternative embodiment, the composition is a solution or suspension, which is dispensed using a pressurised metered dose inhaler (pMDI). The composition according to this embodiment can comprise the dry powder composition discussed above, mixed with or dissolved in a liquid propellant such as HFA134a or HFA227.

It is anticipated that the delivery of an antidepressant via pulmonary inhalation will be more efficient than delivery by the oral route used at present. It is also suggested that this efficient delivery will allow the dosing levels to be reduced and that reduced side effects may also be observed.

The dosing efficiency is expected to lead to a clinical effect being observed following administration by inhalation of doses of an antidepressant which are lower than the doses required to achieve the same therapeutic effect when the antidepressant is administered orally. For example, whilst it has been disclosed that PE may be treated with oral doses of clomipramine starting at 25 mg to 50 mg, it is anticipated that clomipramine doses of less than about 25 mg, and preferably of less than about 20, about 15, about 10 or about 5 mg will be effective when administered by pulmonary inhalation. In one embodiment of the present invention, the dose of an antidepressant administered by pulmonary inhalation is between about 0.1 and about 20 mg, between about 0.2 and about 15 mg, between about 0.5 and about 10 mg, or between about 1 and about 5 mg. Other preferred ranges for pulmonary doses of clomipramine or other antidepressants include about 0.1 to about 5 mg, about 0.2 to about 5 mg and about 0.5 to about 5 mg.

In some embodiments of the present invention, the antidepressant comprises from about 1% to about 99%, from about 3% to about 80%, from about 5% to about 50%, or from about 15% to about 40% of the powder composition.

According to another aspect, the present invention provides unit doses of the antidepressant for treating premature ejaculation. The unit doses comprise the pharmaceutical compositions comprising an antidepressant discussed above.

In one embodiment, blisters are provided containing the compositions according to the present invention. The blisters are preferably foil blisters and comprise a base having a cavity formed therein, the cavity containing a powder composition, the cavity having an opening which is sealed by a rupturable covering.

The doses and/or drug loaded blisters preferably include from about 0.1 to about 20 mg of the powder composition, more preferably about 1 to about 5 mg of the powder composition, wherein the antidepressant comprises from about 1 to about 99%, from about 3% to about 80%, from about 5% to about 50%, or from about 15% to about 40% of the powder composition.

According to another aspect of the present invention, a dry powder inhaler device is provided, comprising a composition according to the invention, as described herein.

In one embodiment, the inhaler is an active inhaler. In another embodiment, the inhaler is a breath actuated inhaler device.

In one embodiment, the composition according to the present invention is held in a blister, the contents of which may be dispensed using one of the aforementioned devices. Preferably, the blister is a foil blister. In another embodiment, the blister comprises polyvinyl chloride or polypropylene in contact with the composition.

According to yet another aspect, the present invention provides methods for producing an inhalable aerosol of a powdered antidepressant composition, according to the first aspect of the invention.

According to another aspect of the present invention, there is provided the use of an antidepressant in the manufacture of a medicament for treating premature ejaculation by pulmonary inhalation. In one embodiment, the antidepressant is a tricyclic antidepressant, such as clomipramine. The medicament may be a composition according to the first aspect of the present invention.

Although certain of the compositions, methods of treatment, inhalers, blisters, methods for inhaling, and doses have been described above as including a carrier material having a preferred average particle size of from about 40 μm to about 70 μm, it should be appreciated that, in accordance with other embodiments, the carrier material in these compositions, methods or treatment, inhalers, blisters, methods for inhaling, and doses can have other average particle size ranges, for example, from about 5 μm to about 1000 μm, from about 10 μm to about 70 μm, from about or from about 20 μm to about 30 μm.

The present invention provides a number of significant advantages over the prior art. In particular, the present invention provides high performance pulmonary delivery of antidepressants, enabling them to be used for reliable, convenient and efficient treatment of PE. This high performance should enable rapid peak blood levels to be achieved and provide rapid clinical onset of the therapeutic effect. The effect of the pulmonary administration of an antidepressant provided by the present invention is consistent and reproducible and this consistency of the high performance administration leads to a reduction in the side effects normally associated with the administration of such agents. The consistent high performance also requires a lower total dose compared to that which would be required if other routes of administration were used.

In addition, the present invention also provides a shorter duration of effect following pulmonary administration, which is expected to further reduce the adverse side effects experienced by the subject.

### BRIEF DESCRIPTION OF THE DRAWINGS

FIG. 1 shows schematically a preferred inhaler that can be used to deliver the powder formulations according to the present invention.

FIG. 2 shows an asymmetric vortex chamber which may be used in an inhaler device used to dispense the powder formulations of the present invention.

FIG. 3 shows a sectional view of an alternative form of vortex chamber from an asymmetric inhaler.

### DETAILED DESCRIPTION OF THE PREFERRED EMBODIMENTS

The inhalable formulations in accordance with the present invention are preferably administered via a dry powder inhaler (DPI), but can also be administered via a pressurized metered dose inhaler (pMDI), or even via a nebulised system.

Dry Powder Inhaler Formulations

It is known to administer pharmaceutically active agents to a patient by pulmonary administration of a particulate medicament composition which includes the active agent in the form of fine, dry particles (active particles). The size of the active particles is of great importance in determining the site of absorption of the active agent in the lung. In order for the particles to be carried deep into the lungs, the particles must be very fine, for example having a mass median aerodynamic diameter (MMAD) of less than 10 μm. Particles having aerodynamic diameters greater than about 10 μm are likely to impact the walls of the throat and generally do not reach the lung. Particles having aerodynamic diameters in the range of about 5 μm to about 2 μm will generally be deposited in the respiratory bronchioles whereas smaller particles having aerodynamic diameters in the range of about 3 to about 0.05 μm are likely to be deposited in the alveoli.

In one embodiment of the present invention, the composition comprises active particles comprising an antidepressant, the active particles having an MMAD of no more than about 10 μm. In another embodiment, the active particles have an MMAD of from about 5 μm to about 2 μm. In yet another embodiment, the active particles have aerodynamic diameters in the range of about 3 to about 0.05 μm. In one embodiment of the invention, at least 90% of the active particles have a particle size of 5 μm or less. The active agent in the particles is to be absorbed into the bloodstream as quickly as possible, to provide a rapid therapeutically effective blood plasma level of the active agent. Thus, the active particles preferably have a particle size of about 5 μm or less.

Particles having a diameter of less than about 10 μm are, however, thermodynamically unstable due to their high surface area to volume ratio, which provides significant excess surface free energy and encourages particles to agglomerate. In the inhaler, agglomeration of small particles and adherence of particles to the walls of the inhaler are problems that result in the active particles leaving the inhaler as large agglomerates or being unable to leave the inhaler and remaining adhered to the interior of the device, or even clogging or blocking the inhaler.

The uncertainty as to the extent of formation of stable agglomerates of the particles between each actuation of the inhaler, and also between different inhalers and different batches of particles, leads to poor dose reproducibility. Furthermore, the formation of agglomerates means that the MMAD of the active particles can be vastly increased, with agglomerates of the active particles not reaching the required part of the lung. Consequently, it is an aim of the present invention to provide a powder formulation which provides good reproducibility and therefore accurate and predictable dosing.

The metered dose (MD) of a dry powder formulation is the total mass of active agent present in the metered form presented by the inhaler device in question. For example, the MD might be the mass of active agent present in a capsule for a Cyclohaler™, or in a foil blister in an Aspirair™ device.

The emitted dose (ED) is the total mass of the active agent emitted from the device following actuation. It does not include the material left inside or on the surfaces of the device. The ED is measured by collecting the total emitted mass from the device in an apparatus frequently referred to as a dose uniformity sampling apparatus (DUSA), and recovering this by a validated quantitative wet chemical assay.

The fine particle dose (FPD) is the total mass of active agent which is emitted from the device following actuation which is present in an aerodynamic particle size smaller than a defined limit. Where the term fine particle dose or FPD is used herein, the aerodynamic particle size is smaller than 5 μm. The FPD is measured using an impactor or impinger, such as a twin stage impinger (TSI), multi-stage liquid impinger (MSLI), Andersen Cascade Impactor (ACI) or a Next Generation Impactor (NGI). Each impactor or impinger has a pre-determined aerodynamic particle size collection cut point for each stage. The FPD value is obtained by interpretation of the stage-by-stage active agent recovery quantified by a validated quantitative wet chemical assay where either a simple stage cut is used to determine FPD or a more complex mathematical interpolation of the stage-by-stage deposition is used.

The fine particle fraction (FPF) is normally defined as the FPD divided by the ED and expressed as a percentage. Herein, the term percent fine particle dose (% FPD) is used to mean the percentage of the total metered dose which is delivered with a diameter of not more than 5 μm (i.e., % FPD=100*FPD/total metered dose).

The term “ultrafine particle dose” (UFPD) is used herein to mean the total mass of active material delivered by a device which has a diameter of not more than 3 μm. The term “ultrafine particle fraction” is used herein to mean the percentage of the total amount of active material delivered by a device which has a diameter of not more than 3 μm. The term percent ultrafine particle dose (% UFPD) is used herein to mean the percentage of the total metered dose which is delivered with a diameter of not more than 3 μm (i.e., % UFPD=100*UFPD/total metered dose).

The terms “delivered dose” and “emitted dose” or “ED” are used interchangeably herein. These are measured as set out in the current EP monograph for inhalation products.

“Actuation of an inhaler” refers to the process during which a dose of the powder is removed from its rest position in the inhaler. That step takes place after the powder has been loaded into the inhaler ready for use.

The tendency of fine particles to agglomerate means that the FPF of a given dose can be highly unpredictable and a variable proportion of the fine particles will be administered to the lung, or to the correct part of the lung, as a result. This is observed, for example, in formulations comprising pure drug in fine particle form. Such formulations exhibit poor flow properties and poor FPF under most circumstances.

In an attempt to improve this situation and to provide a consistent FPF and FPD, dry powder formulations often include additive material.

The additive material is intended to reduce the cohesion between particles in the dry powder formulation. It is thought that the additive material interferes with the weak bonding forces between the small particles, helping to keep the particles separated and reducing the adhesion of such particles to one another, to other particles in the formulation if present and to the internal surfaces of the inhaler device. Where agglomerates of particles are formed, the addition of particles of additive material decreases the stability of those agglomerates so that they are more likely to break up in the turbulent air stream created on actuation of the inhaler device, whereupon the particles are expelled from the device and inhaled. As the agglomerates break up, the active particles may return to the form of small individual particles or agglomerates of small numbers of particles which are capable of reaching the lower lung.

In the prior art, dry powder formulations are discussed which include distinct particles of additive material (generally of a size comparable to that of the fine active particles). In some embodiments, the additive material may form a coating, generally a discontinuous coating, on the active particles and/or on any carrier particles.

Preferably, the additive material is an anti-adherent material and it will tend to reduce the cohesion between particles and will also prevent fine particles becoming attached to surfaces within the inhaler device. Advantageously, the additive material is an anti-friction agent or glidant and will give the powder formulation better flow properties in the inhaler. The additive materials used in this way may not necessarily be usually referred to as anti-adherents or anti-friction agents, but they will have the effect of decreasing the cohesion between the particles or improving the flow of the powder. The additive materials are sometimes referred to as force control agents (FCAs) and they usually lead to better dose reproducibility and higher FPFs.

Therefore, an additive material or FCA, as used herein, is a material whose presence on the surface of a particle can modify the adhesive and cohesive surface forces experienced by that particle, in the presence of other particles and in relation to the surfaces that the particles are exposed to. In general, its function is to reduce both the adhesive and cohesive forces.

The reduced tendency of the particles to bond strongly, either to each other or to the device itself, not only reduces powder cohesion and adhesion, but can also promote better flow characteristics. This leads to improvements in the dose reproducibility because it reduces the variation in the amount of powder metered out for each dose and improves the release of the powder from the device. It also increases the likelihood that the active material, which does leave the device, will reach the lower lung of the patient.

It is favourable for unstable agglomerates of particles to be present in the powder when it is in the inhaler device. As indicated above, for a powder to leave an inhaler device efficiently and reproducibly, the particles of such a powder should be large, preferably larger than about 40 μm. Such a powder may be in the form of either individual particles having a size of about 40 μm or larger and/or agglomerates of finer particles, the agglomerates having a size of about 40 μm or larger. The agglomerates formed can have a size of as much as about 1000 μm and, with the addition of the additive material, those agglomerates are more likely to be broken down efficiently in the turbulent airstream created on inhalation. Therefore, the formation of unstable or “soft” agglomerates of particles in the powder may be favoured compared with a powder in which there is substantially no agglomeration. Such unstable agglomerates are stable whilst the powder is inside the device but are then disrupted and broken up when the powder is dispensed.

The reduction in the cohesion and adhesion between the active particles can lead to equivalent performance with reduced agglomerate size, or even with individual particles.

Thus, in another embodiment of the present invention, the composition comprises active particles and an additive material. The additive material may be in the form of particles which tend to adhere to the surfaces of the active particles, as disclosed in WO 97/03649. Alternatively, the additive material may be coated on the surface of the active particles by, for example a co-milling method as disclosed in WO 02/43701. Co-spray drying is another method of producing active particles with an additive material on their surfaces. Other possible methods of manufacturing such “coated” active particles include supercritical fluid processing, spray-freeze drying, various forms of precipitation and crystallisation from bulk solution, and other methods which would be well-known to the person skilled in the art.

In certain embodiments of the present invention, the formulation is a “carrier free” formulation, which includes only the antidepressant and one or more additive materials and no carrier or excipient materials. Such carrier free formulations are described in WO 97/03649, the entire disclosure of which is hereby incorporated by reference.

The powder includes at least 60% by weight of the antidepressant, based on the weight of the powder. Advantageously, the powder comprises at least 70%, more preferably at least 80% by weight of the antidepressant. Most advantageously, the powder comprises at least 90%, more preferably at least 95%, more preferably at least 97%, by weight of the antidepressant, based on the weight of the powder.

It is believed that there are physiological benefits in introducing as little powder as possible to the lungs, in particular material other than the active ingredient to be administered to the patient. Therefore, the quantities in which the additive material is added are preferably as small as possible. The most preferred powder, therefore, would comprise more than 99% by weight of the antidepressant.

Advantageously, in these “carrier free” formulations, at least 90% by weight of the particles of the powder have a particle size less than 63 μm, preferably less than 30 μm and more preferably less than 10 μm. As indicated above, the size of the active particles of the powder should be within the range of from about 0.1 μm to about 5 μm for effective delivery to the lower lung. Where the additive material is in particulate form, it may be advantageous for these additive particles to have a size outside the preferred range for delivery to the lower lung.

It is particularly advantageous for the additive material to comprise an amino acid. Amino acids have been found to give, when present as additive material, high respirable fraction of the active material and also good flow properties of the powder. A preferred amino acid is leucine, in particular L-leucine. Although the L-form of the amino acids is generally preferred, the D- and DL-forms may also be used. The additive material may comprise one or more of any of the following amino acids: leucine, isoleucine, lysine, valine, methionine, cysteine, and phenylalanine. Advantageously, the powder includes at least 80%, preferably at least 90% by weight of the active agent, based on the weight of the powder. Advantageously, the powder includes not more than 8%, more advantageously not more than 5% by weight of additive material based on the weight of the powder. As indicated above, in some cases it will be advantageous for the powder to contain about 1% by weight of additive material.

In an alternative embodiment, the additive material includes magnesium stearate or colloidal silicon dioxide.

The additive material or FCA may be provided in an amount from about 0.1% to about 50% by weight, and preferably from about 0.15% to about 30%, from about 0.2 to about 20%, from about 0.25% to about 15%, from about 0.5% to about 10%, from about 0.5% to about 5%, or from about 0.5% to about 2% by weight. In the context of the present invention, suitable additive materials include, but are not limited to, anti-adherent materials. Additive materials may include, for example, magnesium stearate, leucine, lecithin, and sodium stearyl fumarate, and are described more fully in WO 96/23485, which is hereby incorporated by reference.

When the additive material is micronised leucine or lecithin, it is preferably provided in an amount from about 0.1% to about 10% by weight. Preferably, the additive material comprises from about 3% to about 7%, preferably about 5%, of micronised leucine. Preferably, at least 95% by weight of the micronised leucine has a particle diameter of less than 150 μm, preferably less than 100 μm, and most preferably less than 50 μm. Preferably, the mass median diameter of the micronised leucine is less than 10 μm.

If magnesium stearate or sodium stearyl fumarate is used as the additive material, it is preferably provided in an amount from about 0.05% to about 10%, from about 0.15% to about 5%, from about 0.25% to about 2%, or from about 0.15% to about 0.5%.

In a further attempt to improve extraction of the dry powder from the dispensing device and to provide a consistent FPF and FPD, dry powder formulations often include coarse carrier particles of excipient material mixed with fine particles of active material. Rather than sticking to one another, the fine active particles tend to adhere to the surfaces of the coarse carrier particles whilst in the inhaler device, but are supposed to release and become dispersed upon actuation of the dispensing device and inhalation into the respiratory tract, to give a fine suspension. The carrier particles preferably have MMADs greater than about 60 μm or greater than about 40 μm.

The inclusion of coarse carrier particles is also very attractive where very small doses of active agent are dispensed. It is very difficult to accurately and reproducibly dispense very small quantities of powder and small variations in the amount of powder dispensed will mean large variations in the dose of active agent where only very small amounts of the powder is dispensed and the powder comprises mainly active particles. Therefore, the addition of a diluent, in the form of large excipient particles will make dosing more reproducible and accurate.

Carrier particles may be of any acceptable inert excipient material or combination of materials. For example, the carrier particles may be composed of one or more materials selected from sugar alcohols, polyols and crystalline sugars. Other suitable carriers include inorganic salts such as sodium chloride and calcium carbonate, organic salts such as sodium lactate and other organic compounds such as polysaccharides and oligosaccharides. Advantageously, the carrier particles comprise a polyol. In particular, the carrier particles may be particles of crystalline sugar, for example mannitol, dextrose or lactose. Preferably, the carrier particles are composed of lactose.

However, a further difficulty which may be encountered when adding coarse carrier particles to a composition of fine active particles is ensuring that the fine particles detach from the surface of the relatively large carrier particles upon actuation of the delivery device.

The step of dispersing the active particles from other active particles and from carrier particles, if present, to form an aerosol of fine active particles for inhalation is significant in determining the proportion of the dose of active material which reaches the desired site of absorption in the lungs. In order to improve the efficiency of that dispersal it is known to include in the composition additive materials of the nature discussed above. Compositions comprising fine active particles carrier particles and additive materials are disclosed in WO 96/23485.

Thus, in one embodiment of the present invention, the composition comprises active particles and carrier particles. The carrier particles may have an average particle size of from about 5 to about 1000 μm, from about 4 to about 40 μm, from about 60 to about 200 μm, or from 150 to about 1000 μm. Other useful average particle sizes for carrier particles are about 20 to about 30 μm or from about 40 to about 70 μm.

The composition comprising an antidepressant and carrier particles may further include additive material. The additive material may be in the form of particles which tend to adhere to the surfaces of the active particles, as disclosed in WO 97/03649. Alternatively, the additive material may be coated on the surface of the active particles by, for example a co-milling method as disclosed in WO 02/43701 or on the surfaces of the carrier particles, as disclosed in WO 02/00197.

In a dry powder inhaler, the dose to be administered is stored in the form of a non-pressurized dry powder and, on actuation of the inhaler, the particles of the powder are inhaled by the patient. Dry powder inhalers can be “passive” devices in which the patient's breath is the only source of gas which provides a motive force in the device. Examples of “passive” dry powder inhaler devices include the Rotahaler and Diskhaler (GlaxoSmithKline) and the Turbohaler (Astra-Draco) and Novolizer™ (Viatris GmbH). Alternatively, “active” devices may be used, in which a source of compressed gas or alternative energy source is used. Examples of suitable active devices include Aspirair™ (Vectura Ltd) and the active inhaler device produced by Nektar Therapeutics (as covered by U.S. Pat. No. 6,257,233).

Particularly preferred “active” dry powder inhalers are referred to herein as Aspirair inhalers and are described in more detail in WO 01/00262, WO 02/07805, WO 02/89880 and WO 02/89881, the contents of which are hereby incorporated by reference. It should be appreciated, however, that the compositions of the present invention can be administered with either passive or active inhaler devices.

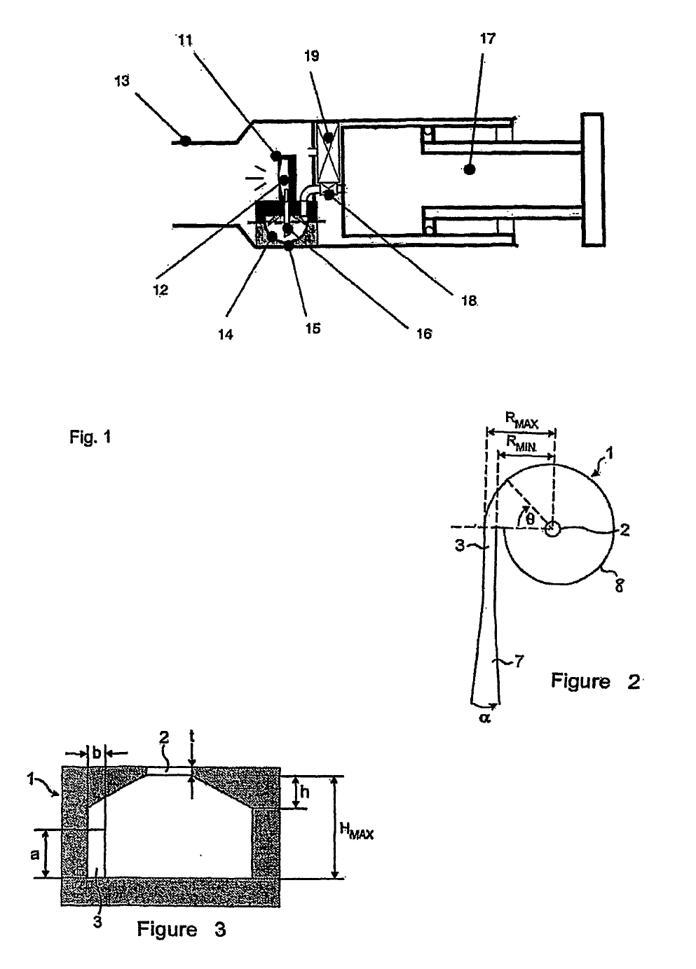

FIG. 1 shows schematically a preferred inhaler that can be used to deliver the powder formulations described above to a patient. Inhalers of this type are described in detail in WO 02/089880 and WO 02/089881.

Referring to FIGS. 1 and 2, the inhaler comprises a vortex nozzle 11 including a vortex chamber 12 and having an exit port and an inlet port for generating an aerosol of the powder formulation. The vortex chamber is located in a mouthpiece 13 through which the user inhales to use the inhaler. Air passages (not shown) may be defined between the vortex chamber and the mouthpiece so that the user is able to inhale air in addition to the powdered medicament.

The powder formulation is stored in a blister 14 defined by a support and a pierceable foil lid. A blister holder 15 holds the blister in place. As shown, the support has a cavity formed therein for holding the powder formulation. The open end of the cavity is sealed by the lid. An air inlet conduit of the vortex chamber terminates in a piercing head 16 which pierces the pierceable foil lid. A reservoir 17 is connected to the blister via a passage. An air supply, preferably a manually operated pump or a canister of pressurized gas or propellant, charges the reservoir with a gas (e.g., air, in this example) to a predetermined pressure (e.g. 1.5 bar). In a preferred embodiment the reservoir comprises a piston received in a cylinder defining a reservoir chamber. The piston is pushed into the cylinder to reduce the volume of the chamber and pressurize the charge of gas.

When the user inhales, a valve 18 is opened by a breath-actuated mechanism 19, forcing air from the pressurized air reservoir through the blister where the powdered formulation is entrained in the air flow. The air flow transports the powder formulation to the vortex chamber 12, where a rotating vortex of powder formulation and air is created between the inlet port and the outlet port. Rather than passing through the vortex chamber in a continuous manner, the powdered formulation entrained in the airflow enters the vortex chamber in a very short time (typically less than 0.3 seconds and preferably less than 20 milliseconds) and, in the case of a pure drug formulation (i.e., no carrier), a portion of the powder formulation sticks to the walls of the vortex chamber. This powder is subsequently aerosolized by the high shear forces present in the boundary layer adjacent to the powder. The action of the vortex deagglomerates the particles of powder formulation, or in the case of a formulation comprising a drug and a carrier, strips the drug from the carrier, so that an aerosol of powdered formulation exits the vortex chamber via the exit port. The aerosol is inhaled by the user through the mouthpiece.

The vortex chamber can be considered to perform several functions, including: deagglomeration, the breaking up of clusters of particles into individual, respirable particles; and filtration, preferentially allowing particles below a certain size to escape more easily from the exit port. Deagglomeration breaks up cohesive clusters of powdered formulation into respirable particles, and filtration increases the residence time of the clusters in the vortex chamber to allow more time for them to be deagglomerated. Deagglomeration can be achieved by turbulence and by creating high shear forces due to velocity gradients in the airflow in the vortex chamber. The velocity gradients are highest in the boundary layer close to the walls of the vortex chamber.

The vortex chamber is in the form of a substantially cylindrical chamber. Advantageously, the vortex chamber has an asymmetric shape. In the embodiment shown in FIGS. 2 and 3, the wall 8 of the vortex chamber is in the form of a spiral or scroll. The inlet port 3 is substantially tangential to the perimeter of the vortex chamber 1 and the exit port 2 is generally concentric with the axis of the vortex chamber 1. Thus, gas enters the vortex chamber 1 tangentially via the inlet port 3 and exits axially via the exit port 2. The radius R of the vortex chamber 1 measured from the center of the exit port 2 decreases smoothly from a maximum radius Rₘₐₓ at the inlet port to a minimum radius Rₘᵢₙ. Thus, the radius R at an angle θ (theta) from the position of the inlet port 3 is given by R=Rₘₐₓ(1−θk/2pi), where k=(Rₘₐₓ−Rₘᵢₙ)/Rₘₐₓ. The effective radius of the vortex chamber 1 decreases as the air flow and entrained particles of medicament circulate around the chamber. In this way, the effective cross-sectional area of the vortex chamber 1 experienced by the air flow decreases, so that the air flow is accelerated and there is reduced deposition of the entrained particles of medicament. In addition, when the flow of air has gone through 2pi radians (360°), the air flow is parallel to the incoming airflow through the inlet port 3, so that there is a reduction in the turbulence caused by the colliding flows which helps reduce fluid losses in the vortex.

Between the inlet port 3 and the exit port 2 a vortex is created in which shear forces are generated to deagglomerate the particles of the powdered formulation. The length of the exit port 2 is preferably as short as possible to reduce the possibility of deposition of the drug on the walls of the exit port. FIG. 3 shows the general form of the vortex chamber of the inhaler of FIG. 2. The geometry of the vortex chamber is defined by the dimensions listed in the table below. The preferred values of these dimension are also listed in the table. It should be noted that the preferred value of the height h of the conical part of the chamber is 0 mm, because it has been found that the vortex chamber functions most effectively when the top (roof) of the chamber is flat.

| | | Preferred |
|---|---|---|
| Dimension | | Value |
| Rₘₐₓ | Maximum radius of chamber | 2.8 mm |
| Rₘᵢₙ | Minimum radius of chamber | 2.0 mm |
| Hₘₐₓ | Maximum height of chamber | 1.6 mm |
| h | Height of conical part of chamber | 0.0 mm |
| Dₑ | Diameter of exit port | 0.7 mm |
| t | Length of exit port | 0.3 mm |
| a | Height of inlet port | 1.1 mm |
| b | Width of inlet port | 0.5 mm |
| α | Taper angle of inlet conduit | 9°, then 2° |

The ratio of the diameter of the chamber 1 to the diameter of the exit port 2 has a strong influence on the aerosolizing performance of the nozzle. For the asymmetric nozzle of FIG. 2, the diameter is defined as (Rₘₐₓ+Rₘᵢₙ). The ratio is between 4 and 12 and preferably between 6 and 8. In the preferred embodiment of FIGS. 2 and 3, the ratio is 6.9.

In the embodiment shown, the vortex chamber is machined from polyetheretherketone (PEEK), acrylic, or brass, although a wide range of alternative materials is possible. Advantageously for high volume manufacture the vortex chamber is injection moulded from a polymer. Suitable materials include but are not limited to polycarbonate, acrylonitrile butadiene styrene (ABS), polyamides, polystyrenes, polybutylene terphthalate (PBT) and polyolefins including polypropylene and polyethylene terephthalate (PET).

The inhaler in accordance with embodiments of the invention is able to generate a relatively slow moving aerosol with a high fine particle fraction. The inhaler is capable of providing complete and repeatable aerosolisation of a measured dose of powdered drug and of delivering the aerosolised dose into the patient's inspiratory flow at a velocity less than or substantially equal to the velocity of the inspiratory flow, thereby reducing deposition by impaction in the patients mouth. Furthermore, the efficient aerosolising system allows for a simple, small and low cost device, because the energy used to create the aerosol is small. The fluid energy required to create the aerosol can be defined as the integral over time of the pressure multiplied by the flow rate. This is typically less than 5 joules and can be as low as 3 joules.

In certain embodiments of the present invention, the powder composition is such that a fine particle fraction of at least 35% is generated on actuation of the inhaler device. It is particularly preferred that the fine particle fraction be greater than or equal to 45%, 50% or 60%. Preferably, the fine particle fraction is at least 70%, and most preferably at least 80%. In one embodiment, this powder comprises an antidepressant in combination with a carrier material.

Most preferably, the inhaler device used to dispense the powder composition is an active inhaler device, the arrangement being such that a fine particle fraction of at least 35%, preferably at least 50%, even more preferably at least 60%, even more preferably at least 70%, and most preferably at least 80% is generated on actuation of the inhaler device. As an active device does not depend on the patient's inhalation for aerosolising the dose, the delivery of the dose is more repeatable than is observed using passive inhaler devices.

In accordance with another embodiment of the present invention, the dose of active agent is defined in terms of the fine particle dose of the administered dose. The percentage of the antidepressant in the dose which will reach the lung (the % FPD) is dependent on the formulation used and on the inhaler used. As such, a 10 mg dose of the antidepressant, for example clomipramine, will deliver 3.5 mg of clomipramine to the lung of a patient if a % FPD of 35% is achieved, whilst the same dose will deliver 6 mg of clomipramine to the lung of a patient if a % FPD of 60% is achieved, or 7 mg if the % FPD is 70%, as anticipated in the present invention. As such, it is appropriate to define the dose of antidepressant in terms of the FPD of the formulation and inhaler used, as measured by a Multistage Liquid Impinger or an Anderson Cascade Impactor.

As such, in accordance with another embodiment of the present invention, a method for treating premature ejaculation via inhalation is provided which comprises inhaling a dose of a powder composition into the lungs of a patient, the dose of the powder composition delivering, in vitro, a fine particle dose of a fine particle dose of from about 0.1 mg to about 20 mg of an antidepressant, when measured by a Multistage Liquid Impinger, United States Pharmacopoeia 26, Chapter 601, Apparatus 4 (2003), an Andersen Cascade Impactor or a New Generation Impactor.

The dose of active agent, defined in the manner above in connection with the Multistage Liquid Impinger, can similarly be used in connection with the blisters, inhalers, and compositions described herein.

In addition to the fine particle fraction, another parameter of interest is the ultrafine particle fraction defined above. Although particles having a diameter of less than 5 μm (corresponding to the FPF) are suitable for local delivery to the lungs, it is believed that for systemic delivery, even finer particles are needed, because the drug must reach the alveoli to be absorbed into the bloodstream. As such, it is particularly preferred that the formulations and devices in accordance with the present invention be sufficient to provide an ultrafine particle fraction of at least about 50%, more preferably at least about 60% and most preferably at least about 70%.

Preferably, at least 90% by weight of the active material has a particle size of not more than 10 μm, most preferably not more than 5 μm. The particles therefore give a good suspension on actuation of the inhaler.

According to an embodiment of the present invention, an active inhaler device may be used to dispense the dry powder formulations, in order to ensure that the best fine particle fraction and fine particle dose is achieved and, very importantly, that this is achieved consistently. Preferably, the inhaler device includes a breath triggering means such that the delivery of the dose is triggered by the onset of the patient's inhalation. This means that the patient does not need to coordinate their inhalation with the actuation of the inhaler device and that the dose can be delivered at the optimum point in the inspiratory flow. Such devices are commonly referred to as “breath actuated”.

In embodiments of the present invention which utilize conventional inhalers, such as the Rotohaler and Diskhaler described above, the particle size of the carrier particles may range from about 10 to about 1000 μm. In certain of these embodiments, the particle size of the carrier particles may range from about 20 μm to about 120 μm. In certain other ones of these embodiments, the size of at least to 90% by weight of the carrier particles is less than 1000 μm and preferably lies between 60 μm and 1000 μm. The relatively large size of these carrier particles gives good flow and entertainment characteristics.

In these embodiments, the powder may also contain fine particles of an excipient material, which may for example be a material such as one of those mentioned above as being suitable for use as a carrier material, especially a crystalline sugar such as dextrose or lactose. The fine excipient material may be of the same or a different material from the carrier particles, where both are present. The particle size of the fine excipient material will generally not exceed 30 μm, and preferably does not exceed 20 μm.

The powders may also be formulated with additional excipients to aid delivery and release. For example, as discussed above, powder compositions may be formulated with relatively large carrier particles, for example those having a mass median aerodynamic diameter of greater than 30 μm, greater than 40 μm, greater than 60 μm, or even greater than 90 μm, which aid the flow properties of the powder. Alternatively or additionally, hydrophobic microparticles may be included in the compositions of the present invention. Preferred hydrophobic materials include solid state fatty acids such as oleic acid, lauric acid, palmitic acid, stearic acid, erucic acid, behenic acid, or derivatives (such as esters and salts) thereof. Specific examples of such materials include phosphatidylcholines, phosphatidylglycerols and other examples of natural and synthetic lung surfactants. Particularly preferred materials include metal stearates, in particular magnesium stearate, which has been approved for delivery via the lung.

Large carrier particles are particularly useful when they are included in compositions which are to be dispensed using a passive inhaler device, such as the Diskhaler and Rotahaler devices discussed above. These devices do not create high turbulence within the device upon actuation and so the presence of the carrier particles is beneficial as they have a beneficial effect on the flow properties of the powder, making it easier to extract the powder from the blister or capsule within which it is stored.

In some circumstances, the powder for inhalation may be prepared by mixing the components of the powder together. For example, the powder may be prepared by mixing together particles of active material and lactose.

In embodiments of the present invention which utilize an active inhaler, for example an Aspirair inhaler as described above, the carrier particles are preferably between 5 and 100 μm, and may be between 40 and 70 μm in diameter or between 20 and 30 μm in diameter. The desired particle size can be achieved for example, by sieving the excipient. For a desired particle size range of between 40 and 70 μm, the material may be sieved through screens of 45 μm and 63 μm, thereby excluding particles that pass through the 45 μm screen, and excluding particles that do not pass through the 63 μm screen. Most preferably, the excipient is lactose.

Preferably, at least 90%, and most preferably at least 99%, of the active particles are 5 μm or less in diameter. As detailed below, such a formulation, when administered via the preferred active inhalers, can provide a fine particle fraction in excess of about 80%, and an ultrafine particle fraction in excess of about 70%.

In such formulations where the dispensing device creates high turbulence within the device upon actuation, the powder does not need to include large carrier particles to enhance the flow properties of the powder. The device is capable of extracting powders even if they have poor flow properties and so the diluent material used in such formulations can have a smaller particle size. In one embodiment, the particles of excipient material may even be 10 μm in diameter or less.

The dry powder inhaler devices in which the powder compositions of the present invention will commonly be used include “single dose” devices, for example the Rotahaler™ and the Spinhaler™ in which individual doses of the powder composition are introduced into the device in, for example, single dose capsules or blisters, and also multiple dose devices, for example the Turbohaler™ in which, on actuation of the inhaler, one dose of the powder is removed from a reservoir of the powder material contained in the device.

As already mentioned, in the case of certain powders, an active inhaler device offers advantages in that a higher fine particle fraction and a more consistent dose to dose repeatability will be obtainable than if other forms of device were used. Such devices include, for example, the Aspirair™ or the Nektar Therapeutics active inhaler device, and may be breath actuated devices of the kind in which generation of an aerosolised cloud of powder is triggered by inhalation of the patient.

Where present, the amount of carrier particles may be up to 99%, up to 95%, up to 90%, up to 80% or up to 50% by weight based on the total weight of the powder. The amount of any fine excipient material, if present, may be up to 90%, up to 50% and advantageously up to 30%, especially up to 20%, by weight, based on the total weight of the powder.

Where reference is made to particle size of particles of the powder, it is to be understood, unless indicated to the contrary, that the particle size is the volume weighted particle size. The particle size may be calculated by a laser diffraction method. Where the particle also includes an additive material on the surface of the particle, advantageously the particle size of the coated particles is also within the preferred size ranges indicated for the uncoated particles.

While it is clearly desirable for as large a proportion as possible of the particles of active material to be delivered to the deep lung, it is usually preferable for as little as possible of the other components to penetrate the deep lung. Therefore, powders generally include particles of an active material and carrier particles for carrying the particles of active material.

As described in WO 01/82906, an additive material may also be provided in a dose which indicates to the patient that the dose has been administered. The additive material, referred to below as indicator material, may be present in the powder as formulated for the dry powder inhaler, or be present in a separate form, such as in a separate location within the inhaler such that the additive becomes entrained in the airflow generated on inhalation simultaneously or sequentially with the powder containing the active material.

In some circumstances, for example, where any carrier particles and/or any fine excipient material present is of a material itself capable of inducing a sensation in the oropharyngeal region, the carrier particles and/or the fine excipient material can constitute the indicator material. For example, the carrier particles and/or any fine particle excipient may comprise mannitol. Another suitable indicator material is menthol.

In certain embodiments of the present invention, each dose is stored in a foil “blister” of a blister pack. In accordance with the embodiments of the present invention which utilize foil blisters, exposure of the formulation to air prior to administration is reduced or prevented by storing each dose in a sealed foil blister. In some circumstances, it may be desirable to further protect the formulation by placing a plurality of blisters into a further sealed container, such as a sealed bag made, for example of a foil such as aluminium foil. Further mechanical protection may also be desirable, to protect the sealed blisters from damage during storage and transportation, etc. The use of the sealed foil blisters (and optional sealed bags and/or other protective packaging) eliminates any need to include anti-oxidants or the like in the formulation.

The blisters which may be used in the present invention consist of a base and a lid. Preferably, the base material is a laminate comprising a polymer layer in contact with the drug, a soft tempered aluminium layer and an external polymer layer. The aluminium provides the moisture and oxygen barrier, whilst the polymer provides a relatively inert layer in contact with the drug. Soft tempered aluminium is ductile so that it can be “cold formed” into a blister shape. It is typically 45-47 μm thick. The outer polymer layer provides additional strength to the laminate. The lid material is a laminate comprising a heat seal lacquer, a hard rolled aluminium layer (typically 20-30 μm thick) and an external polymer layer. The heat seal lacquer bonds to the polymer layer of the base foil laminate during heat sealing. The aluminium layer is hard rolled to facilitate piercing. Materials for the polymer layer in contact with the drug include polyvinyl chloride (PVC), polypropylene (PP) and polyethylene (PE). The external polymer layer on the base foil is typically oriented polyamide (oPA).

Pressurized Metered Dose Inhaler Formulations

Pressurized metered dose inhalers (pMDI) typically have two components: a canister component in which the drug particles, in this case an antidepressant, are stored under pressure in a suspension or solution form and a receptacle component used to hold and actuate the canister. Typically, a canister will contain multiple doses of the formulation, although it is possible to have single dose canisters as well. The canister component typically includes a valved outlet from which the contents of the canister can be discharged. Aerosol medication is dispensed from the pMDI by applying a force on the canister component to push it into the receptacle component thereby opening the valved outlet and causing the medication to be conveyed from the valved outlet through the receptacle component and discharged from an outlet of the receptacle component. Upon discharge from the canister, the medication is “atomised”, forming an aerosol.

It is intended that the patient coordinate the discharge of aerosolised medication with his inhalation so that the medication particles are entrained in the patient's inspiratory flow and conveyed to the lungs.

Typically, pMDIs use propellants to pressurize the contents of the canister and to propel the medication out of the outlet of the receptacle component. In pMDI inhalers, the formulation is provided in liquid form, and resides within the container along with the propellant. The propellant can take a variety of forms. For example, the propellant can comprise a compressed gas or a liquefied gas. Suitable propellants include CFC (chlorofluorocarbon) propellants such as CFC 11 and CFC 12, as well as HFA (hydrofluoroalkane) propellants such as HFA134a and HFA227. One or more propellants may be used in a given formulation.

In order to better coordinate actuation of the inhaler with inhalation, a breath actuated valve system may be used. Such systems are available, for example, from Baker Norton and 3M. To use such a device, the patient “primes” the device, and then the dose is automatically fired when the patient inhales.

In certain embodiments, the pMDI formulation is either a “suspension” type formulation or a “solution” type formulation, each using a liquefied gas as the propellant. It is believed that the in vivo affect of pMDI formulations will be similar to those of the DPI formulations described above, in terms of time to therapeutic effect and duration of therapeutic effect.

Solution pMDI

Of pMDI technologies, solution pMDIs are believed to be the most appropriate for systemic lung delivery as they offer the finest mist, and can be more easily optimised through modifications to the device. Recently developed valves (e.g. those available from Bespak) also offer payload increases over current systems, meaning that larger systemic doses can potentially be delivered in solution pMDIs than in suspension type pMDIs. Solution pMDI techniques can be used to prepare formulations for delivery of an antidepressant with HFA propellants.

Suspension pMDI

Suspension pMDIs can also be used to deliver an antidepressant to the lungs. However, suspension pMDIs have a number of disadvantages. For example, suspension pMDIs generally deliver lower doses than solution pMDIs and are prone to other issues related to suspensions, e.g., dose inconsistencies, valve blockage, and suspension instabilities (e.g., settling). For these reasons, and others, suspension pMDIs tend to be much more complex to formulate and manufacture than solution pMDIs.

In accordance with one embodiment of the present invention, a suspension pMDI for an antidepressant is provided. Preferably, the propellant of the suspension pMDI is a blend of two commercially available HFA propellants, most preferably HFA227 (1,1,1,2,3,3,3-heptafluoropropane) and HFA134a (1,1,1,2-tetrafluoroethane). In one embodiment, blends of about 60% HFA227 and about 40% HFA134a are used with an antidepressant in a 3M coated (Dupont 3200 200) canister with a Bespak BK630 series 0.22 mm actuator.

Nebulised Systems

Another possible method of administration is via a nebulised system. Such systems include conventional ultrasonic nebulised systems and jet nebulised systems, as well as recently introduced handheld devices such as the Respimat (available from Boehringer Ingelheim) or the AERx (available from Aradigm). In such a system, the antidepressant could be stabilized in a sterile aqueous solution, for example, with antioxidants such as sodium metabisulfite. The doses would be similar to those described above, adjusted to take into consideration the lower percentage of the antidepressant that will reach the lung in a nebulised system. Although these systems can be used, they are clearly inferior to the DPI systems described above, both in terms of efficiency and convenience of use.

### EXAMPLES

### Jet Milling

Various examples illustrating the invention are discussed below. Unless otherwise stated, the inhaler device used in the examples was an Aspirair prototype inhaler made by Vectura Limited.

Formulations were produced from a commercially available clomipramine hydrochloride powder, using the Hosokawa AS50 jet mill. Either the pure drug was passed through the mill or a blend of drug with 5% w/w of a force control agent added. The mill was used with a range of parameters. Primarily, these were injector air pressure, grinding air pressure and powder feed rate.

Formulation 1: The pure clomipramine hydrochloride was passed through the microniser three times, each time with an injector air pressure of 8 bar, grinding air pressure of 1.5 bar and powder feed rate of approximately 1 g/min. Malvern (dry powder) particle size measurement gave a d(50) of 1.2 μm.

Formulation 2: Formulation 1 was pre-blended in a pestle with a spatula with 50% micronised 1-leucine. This blend was further micronised with an injector air pressure of 8 bar, grinding air pressure of 1.5 bar and powder feed rate of approximately 1 g/min. Malvern (dry powder) particle size measurement gave a d(50) of 1.2 μm.

Formulation 3: The pure clomipramine hydrochloride was mictonised with an injector air pressure of 7 bar, grinding air pressure of 5 bar and powder feed rate of approximately 10 g/min. Malvern (dry powder) particle size measurement gave a d(50) of 1.0 μm.

Formulation 4: The pure clomipramine hydrochloride was micronised with an injector air pressure of 7 bar, grinding air pressure of 5 bar and powder feed rate of approximately 10 g/min. This micronised clomipramine was pre-blended in a pestle with a spatula with 5% micronised 1-leucine. This blend was then micronised with an injector air pressure of 7 bar, grinding air pressure of 5 bar and powder feed rate of approximately 10 g/min. Malvern (dry powder) particle size measurement gave a d(50) of 0.95 μm.

Formulation 5: The clomipramine hydrochloride was pre-blended in a pestle with a spatula with 5% magnesium stearate. This blend was micronised with an injector air pressure of 7 bar, grinding air pressure of 5 bar and powder feed rate of approximately 10 g/min. Malvern (dry powder) particle size measurement gave a d(50) of 0.95 μm.

Formulation 6: The pure clomipramine hydrochloride was micronised with an injector air pressure of 7 bar, grinding air pressure of 1 bar and powder feed rate of approximately 1 g/min. Malvern (dry powder) particle size measurement gave a d(50) of 1.8 μm.

This pre-micronised clomipramine hydrochloride was then blended in a pestle with a spatula with 5% micronised 1-leucine. This blend was then micronised with an injector air pressure of 7 bar, grinding air pressure of 1 bar and powder feed rate of approximately 1 g/min. Malvern (dry powder) particle size measurement gave a d(50) of 1.38 μm.

Formulation 7a: The pure clomipramine hydrochloride was micronised with an injector air pressure of 7 bar, grinding air pressure of 1 bar and powder feed rate of approximately 10 g/min. Malvern (dry powder) particle size measurement gave a d(50) of 3.5 μm.

This pre-micronised clomipramine hydrochloride was then blended in a pestle with a spatula with 5% micronised 1-leucine. This blend was then micronised with an injector air pressure of 7 bar, grinding air pressure of 1 bar and powder feed rate of approximately 10 g/min. Malvern (dry powder) particle size measurement gave a d(50) of 2.0 μm.

Formulation 7b: The pure clomipramine hydrochloride was micronised with an injector air pressure of 7 bar, grinding air pressure of 3 bar and powder feed rate of approximately 1 g/min. Malvern (dry powder) particle size measurement gave a d(50) of 1.21 μm.

This pre-micronised clomipramine hydrochloride was then blended in a pestle with a spatula with 5% micronised 1-leucine. This blend was then micronised with an injector air pressure of 7 bar, grinding air pressure of 3 bar and powder feed rate of approximately 1 g/min. Malvern (dry powder) particle size measurement gave a d(50) of 0.99 μm.

Formulation 7c: The pure clomipramine hydrochloride was micronised with an injector air pressure of 7 bar, grinding air pressure of 3 bar and powder feed rate of approximately 10 g/min. Malvern (dry powder) particle size measurement gave a d(50) of 1.6 μm.

This pre-micronised clomipramine hydrochloride was then blended in a pestle with a spatula with 5% micronised 1-leucine. This blend was then micronised with an injector air pressure of 7 bar, grinding air pressure of 3 bar and powder feed rate of approximately 10 g/min. Malvern (dry powder) particle size measurement gave a d(50) of 1.1 μm.

Formulation 8a: The clomipramine hydrochloride was pre-blended in a pestle with a spatula with 5% micronised 1-leucine. This blend was micronised with an injector air pressure of 7 bar, grinding air pressure of 5 bar and powder feed rate of approximately 10 g/min. Malvern (dry powder) particle size measurement gave a d(50) of 1.8 μm.

Formulation 8b: The pure clomipramine was micronised with an injector air pressure of 7 bar, grinding air pressure of 5 bar and powder feed rate of approximately 10 g/min.

This pre-micronised clomipramine hydrochloride was then blended in a pestle with a spatula with 5% magnesium stearate. This blend was then micronised with an injector air pressure of 7 bar, grinding air pressure of 1 bar and powder feed rate of approximately 10 g/min.

This powder was then processed in the Hosokawa MechanoFusion Mini-kit with 1 mm compression gap for 10 minutes. Malvern (dry powder) particle size measurement gave a d(50) of 1.39 μm.

Formulation 8c: The pure clomipramine hydrochloride was micronised with an injector air pressure of 7 bar, grinding air pressure of 5 bar and powder feed rate of approximately 10 g/min.

This pre-micronised clomipramine hydrochloride was then blended in a pestle with a spatula with 5% magnesium stearate. This blend was then micronised with an injector air pressure of 7 bar, grinding air pressure of 1 bar and powder feed rate of approximately 10 g/min. Malvern (dry powder) particle size measurement gave a d(50) of 1.38 μm.

Formulation 8d: The pure clomipramine hydrochloride was micronised with an injector air pressure of 7 bar, grinding air pressure of 5 bar and powder feed rate of approximately 10 g/min. In this case, Malvern (dry powder) particle size measurement gave a d(50) of 1.67 μm.

Malvern particle size distributions show that clomipramine hydrochloride micronised very readily to small particle sizes. For example, Formulation 3 micronised to 1.0 μm with one pass at the relatively high grinding pressure of 5 bar and the higher powder feed rate of 10 g/min.

Reducing the grinding pressure, for example to 1 bar, as with Formulation 6 interim powder, resulted in larger particles (d(50) of approximately 1.8 μm). Intermediate grinding pressure (3 bar) gave an intermediate particle size distribution (d(50) of approximately 1.2 μm as for Formulation 7b interim powder).

Similarly, increasing powder feed rate, for example from 1 to 10 g/min, resulted in larger particles, as can be seen by comparing d(50)s for Formulations 6 and 7a.

The addition of FCA, for example leucine, as in Formulation 8a, appeared to reduce the milling efficiency. However, this change may have been caused by the concomitant improvement in flowability of the original drug powder leading to a small but significant increase in the powder feed rate into the mill. It was observed in other studies that milling efficiency was increasingly sensitive to this powder feed rate as it increased above 10 g/min.

It appeared possible from this series of examples to design the milling parameters to select a particular d(50). For example, a d(50) of approximately 1.4 could be obtained either by repeated low pressure milling and low feed rate (Formulation 6) or by a mix of higher and lower pressure milling at a higher feed rate Formulation 8c).

Approximately 2 mg of each formulation was then loaded and sealed into a foil blister. This was then fired from an Aspirair device into a Next Generation Impactor with air flow set at 60 μl/min. The performance data are summarised in Tables 1, 2 and 3.

TABLE 1

| | MD | DD | FPD | FPF | |
|---|---|---|---|---|---|
| Formulation | (mg) | (mg) | (mg) | (MD) | MMAD |
| 1 | 1.64 | 1.19 | 1.05 | 64 | 1.53 |
| (pure drug, jet-milled at 8/1.5 bar) | | | | | |
| 2 (5% leucine, | 1.55 | 1.32 | 1.19 | 78 | 1.68 |
| jet-milled at 8/1.5 bar) | | | | | |
| 3 | 2.414 | 1.832 | 1.493 | 62 | 1.80 |
| (pure drug, jet-milled at 7/5 bar) | | | | | |
| 4 | 2.120 | 1.624 | 1.474 | 70 | 1.52 |
| (5% leucine, jet-milled at 7/5 bar) | | | | | |
| 5 | 1.737 | 1.519 | 1.390 | 80 | 1.44 |
| (5% MgSt, jet-milled at 7/5 bar) | | | | | |
| 6 | 2.031 | 1.839 | 1.550 | 76 | 1.90 |
| (5% leucine, jet-milled at 7/1 bar) | | | | | |
| 7a | 1.821 | 1.685 | 1.071 | 59 | 2.44 |
| (5% leucine, jet-milled at 7/1 bar) | | | | | |
| 7b | 1.846 | 1.523 | 1.437 | 78 | 1.61 |
| (5% leucine, jet-milled at 7/3 bar) | | | | | |
| 7c | 2.213 | 1.940 | 1.733 | 78 | 1.72 |
| (5% leucine, jet-milled at 7/3 bar) | | | | | |
| 8a | 1.696 | 1.557 | 1.147 | 68 | 2.13 |
| (5% leucine, single pass at 7/5 bar) | | | | | |
| 8b | 1.743 | 1.542 | 1.274 | 73 | 1.82 |
| (5% MgSt, jet-milled at 7/5 bar & | | | | | |
| Mechano-Fused) | | | | | |
| 8c | 1.677 | 1.570 | 1.351 | 81 | 1.72 |
| (5% MgSt, jet-milled at 7/5 bar) | | | | | |
| 8d | 2.049 | 1.755 | 1.447 | 71 | 1.83 |
| (pure drug, jet-milled at 7/5 bar) | | | | | |

TABLE 2

| | FPF % | FPF % | FPF % | FPF % |
|---|---|---|---|---|
| Formulation | (<5 μm) | (<3 μm) | (<2 μm) | (<1 μm) |
| 1 | 88 | 83 | 65 | 21 |
| (pure drug, jet milled at 8/1.5 bar) | | | | |
| 2 | 90 | 82 | 60 | 17 |
| (5% leucine, jet-milled at 8/1.5bar) | | | | |
| 3 | 82 | 71 | 51 | 14 |
| (pure drug, jet-milled at 7/5 bar) | | | | |
| 4 | 91 | 85 | 68 | 21 |
| (5% leucine, jet-milled at 7/5 bar) | | | | |
| 5 | 91 | 90 | 73 | 20 |
| (5% MgSt, jet-milled at 7/5 bar) | | | | |
| 6 | 84 | 74 | 48 | 10 |
| (5% leucine, jet-milled at 7/1 bar) | | | | |
| 7a | 64 | 46 | 28 | 6 |
| (5% leucine, jet-milled at 7/1 bar) | | | | |
| 7b | 94 | 88 | 67 | 14 |
| (5% leucine, jet-milled at 7/3 bar) | | | | |
| 7c | 89 | 80 | 56 | 14 |
| (5% leucine, jet-milled at 7/3 bar) | | | | |
| 8a | 74 | 57 | 37 | 9 |
| (5% leucine, single pass at 7/5 bar) | | | | |
| 8b | 83 | 68 | 47 | 15 |
| (5% MgSt, jet-milled at 7/5 bar & | | | | |
| Mechano-Fused) | | | | |
| 8c | 86 | 74 | 53 | 21 |
| (5% MgSt, jet-milled at 7/5 bar) | | | | |
| 8d | 82 | 69 | 50 | 19 |
| pure drug, jet-milled at 7/5 bar | | | | |

TABLE 3

| Formulation | Recovery % | Throat % | Blister % | Device % |
|---|---|---|---|---|
| 1 | 82 | 8 | 1 | 26 |
| (pure drug, jet milled at 8/1.5 bar) | | | | |
| 2 | 81 | 7 | 0 | 15 |
| (5% leucine, jet-milled at 8/1.5 bar) | | | | |
| 3 | 121 | 10 | 3 | 21 |
| (pure drug, jet-milled at 7/5 bar) | | | | |
| 4 | 106 | 5 | 1 | 23 |
| (5% leucine, jet-milled at 7/5 bar) | | | | |
| 5 | 91 | 6 | 0 | 12 |
| (5% MgSt, jet-milled at 7/5 bar) | | | | |
| 6 | 107 | 10.6 | 1.3 | 8.2 |
| (5% leucine, jet-milled at 7/1 bar) | | | | |
| 7a | 96 | 24 | 1.3 | 6.1 |
| (5% leucine, jet-milled at 7/1 bar) | | | | |
| 7b | 97 | 3 | 0.6 | 16.9 |
| (5% leucine, jet-milled at 7/3 bar) | | | | |
| 7c | 116 | 7 | 0.6 | 16.9 |
| (5% leucine, jet-milled at 7/3 bar) | | | | |
| 8a | 87 | 18 | 2 | 6 |
| (5% leucine, single pass at 7/5 bar) | | | | |
| 8b | 92 | 14 | 1 | 10 |
| (5% MgSt, jet-milled at 7/5 bar & | | | | |
| Mechano-Fused) | | | | |
| 8c | 87 | 10 | 1 | 6 |
| (5% MgSt, jet-milled at 7/5 bar) | | | | |
| 8d | 102 | 9 | 2 | 12 |
| (pure drug, jet-milled at 7/5 bar) | | | | |

The compound appears to have a relatively high tendency to stick in the device cyclone. The device retention appeared high (above 20%) where pure drug was used, and especially increased with small particle sizes (especially 1 μm and below), for example Formulations 1 and 3 had high drug retention. Formulation 8d had a d(59) of 1.8 μm with lower device retention at 12%. Device retention was lower with use of magnesium stearate, for example as with Formulation 5 where device retention was 12% despite a d(50) of 0.95 μm. Device retention was also reduced below 20% when leucine was used in combination with a particle size above 1 μm, for example with Formulation 8a.

Throat deposition was reduced proportionately as particle size was reduced. High throat deposition (>20%) occurs with particle size d(50)>2 μm: e.g. Formulation 7a. Throat deposition of below 10% was seen for particle sizes below 1 μm. The reduced inertial behaviour of the smaller particles may well contribute to this observation. However, as noted above, device retention tended to be greater for such small particles.

It is argued that as particle size was reduced, increased adhesivity and cohesivity results in increased device retention. This adhesivity and cohesivity and hence device retention can be reduced by addition of force control agents, attached to the drug particle surface (or drug and excipients as appropriate). In Aspirair it is believed that a level of adhesivity and cohesivity is desirable to prolong lifetime in the vortex, yielding a slower plume, but adhesivity and cohesivity should not be so high as to result in high device retention. Consequently a balance of particle size, adhesivity and cohesivity is required to achieve an optimum performance in Aspirair.

Single step co-milling with FCA appears effective in some examples such as Formulation 5. It is proposed that multiple stage processing may be more effective where the conditions are selected to achieve particularly desirable effects. For example, first stage high pressure milling of pure drug may be used to produce the required size distribution (i.e. approximately 1.4 μm), and a second stage lower pressure co-milling used to mix in the force control agent, whereby better mixing is achieved without milling and with reduced segregation of components in the mill. This is shown in Formulation 8c, where a combination of both relatively low throat deposition and low device retention are achieved.

Control of particle size from milling appears critical to effective performance in Aspirair. Without the use of FCA it might be possible to get acceptable performance, on condition the d(50) particle size is well controlled within an estimated range of approximately 1.5 to 2 μm. Multiple shots were not fired, hence the tendency for device build-up was not evaluated. However, device retention of >10% on single shots appears high.

Addition of FCA appears to significantly reduce device retention on single shots, with magnesium stearate being more effective than leucine. An optimum performance appears to be for particles in the estimated range of approximately 1.3 to 1.8 μm, which are co-milled with magnesium stearate. In addition, it is suggested that a 2-stage milling may afford improved control, the first to achieve suitable particle size, the second to co-mill at reduced pressure to get coating.

Suitable repeat formulations, repeat tests and attention to issues of dose, recovery, stability and assay would be needed to confirm the above results.

### EXAMPLES

### Spray Drying

An alternative method of preparing fine dry powder particles of an antidepressant is spray drying.

Whilst particles comprising antidepressants may be prepared using conventional spray drying techniques, particularly good performance is observed where the spray drying is adapted to allow the spray dried particles to be “engineered”.

In particular, it has been found that spray dried dry powder formulations exhibit beneficial properties and excellent performance in dry powder inhalers when the spray drying apparatus includes an alternative to the convention two-fluid nozzle to produce the droplets which creates droplets travelling at slower speeds than those created by the two-fluid nozzles. An example of such an alternative droplet forming means is an ultrasonic nebuliser (USN). The spray dried particles formed using a USN tend to be smaller and denser than those formed using a conventional spray drying apparatus. Small particle size distributions have also been observed. What is more, when co-spray drying an active agent with an additive or force control agent, it has been found that the additive can migrate to the surface of the droplet/particle during drying, which makes the additive more effective in controlling particle cohesion as it is present on the surface of the particles.

In this example, formulations comprising clomipramine were prepared by spray drying using an apparatus fitted with an ultrasonic nebuliser. The formulations were tested in Aspirair™ and MonoHaler™ devices.

The clomipramine hydrochloride formulation was produced from an original clomipramine hydrochloride powder, using a spray drying system comprising an ultrasonic nebulisation unit, a gas flow for transporting the droplets nebulised into a heated tube to dry the droplets, and a filtration unit for collecting the dried particles.

An aqueous solution of the clomipramine hydrochloride was made containing 2% w/w relative to the water. Sufficient leucine was added to make 5% w/w relative to the drug.

The solution was nebulised with a frequency of 2.4 MHz and guided through the tube furnace with furnace surface temperature heated to approximately 300° C., after which the dried powder was collected. The gas temperature was not measured, but was substantially less than this temperature. Malvern (dry powder) particle size measurement gave a d(50) of 1.1 μm

The Malvern particle size distributions show that the clomipramine hydrochloride has very small particle sizes and distributions. The d(50) values are 1.1 μm for clomipramine hydrochloride. The mode of the distribution graph is correspondingly 1.15. Further, the spread of the distribution is relatively narrow, with a d(90) value of 2.5 μm, which indicates that substantially all of the powder by mass is less than 3 μm.

Approximately 2 mg of the clomipramine hydrochloride formulation were then loaded and sealed into foil blisters. These were fired from an Aspirair device into a Next Generation Impactor (NGI) with air flow set at 901/min. The results are based upon a single blister shot.

Approximately 20 mg of the clomipramine hydrochloride formulations were loaded and sealed into size 3 capsules. The clomipramine hydrochloride capsules were gelatine capsules. These capsules were then fired using the MonoHaler device into a NGI with an air flow set at 901/min. The performance data are summarised as follows, the data being an average of 2 or 3 determinations:

TABLE 4
Powder performance study of drug and 5% leucine
dispensed using Aspirair (trade mark)

| | | | | | | | FPF |
|---|---|---|---|---|---|---|---|
| | | | | | | | % |
| | MD | DD | FPD | FPF % | FPF % | FPF % | (<1 |
| Aspirair | (μm) | (μm) | (μm) | (<5 μm) | (<3 μm) | (<2 μm) | μm) |
| Clomipramine | 1739 | 1602 | 1461 | 91 | 81 | 62 | 28 |
| 2 mg | | | | | | | |

TABLE 5
Powder performance study of drug and 5% leucine
dispensed using Aspirair (trade mark)

| | | Recovery | Throat | Blister | Device |
|---|---|---|---|---|---|
| Aspirair | MMAD | (%) | (%) | (%) | (%) |
| Clomipramine | 1.56 | 88 | 4 | 3 | 5 |
| 2 mg | | | | | |

TABLE 6
Powder performance study of drug and 5% leucine
dispensed using Monohaler (trade mark)

| | | | FPD | FPF % | FPF % | FPF % | FPF % |
|---|---|---|---|---|---|---|---|
| Monohaler | MD (μm) | DD (μm) | (μm) | (<5 μm) | (<3 μm) | (<2 μm) | (<1 μm) |
| Clomipramine | 18359 | 16441 | 12685 | 77 | 56 | 37 | 19 |
| 20 mg | | | | | | | |

TABLE 7
Powder performance study of drug and 5% leucine
dispensed using Monohaler (trade mark)

| | | Recovery | Throat | Blister | Device |
|---|---|---|---|---|---|
| Monohaler | MMAD | (%) | (%) | (%) | (%) |
| Clomipramine | 2.38 | 86 | 10 | 1 | 9 |
| 20 mg | | | | | |

The device retention in the Aspirair device was surprisingly low at 5%. This was especially low given the small particle sizes used (d(50) of 1.1 μm) and the relatively high dose loadings used. In comparison, clomipramine hydrochloride co-jet milled with 5% leucine with a d(50) of 0.95 μm gave a device retention of 23% under otherwise similar circumstances.

When using the Monohaler device to dispense the formulations, the device retention was higher than observed when the Aspirair device was used. However, device retention of 9% still appears to be relative low for a formulation that comprises >90% ultrafine drug.

Throat retention was also very low. When the formulations were dispensed using the Aspirair, it was as low as 4%, whilst with Monohaler as the device, the results show slightly higher throat retention (10%).

It has previous been argued that as particle size was reduced, powder surface free energy and hence powder adhesivity and cohesivity would increase. This would be expected to result in increased device retention and poor dispersion. Such adhesivity and cohesivity and hence device retention/poor performance has been shown to be reduced by addition of force control agents, attached to the drug particle surface (or drug and excipients as appropriate). In Aspirair, it is believed that a level of adhesivity and cohesivity is desirable to prolong lifetime in the vortex, yielding a slower plume, but adhesivity and cohesivity should not be so high as to result in high device retention. Consequently a balance of particle size, adhesivity and cohesivity is believed to be required to achieve an optimum performance in Aspirair.

The dispersion results for the powder was excellent when using Monohaler as the device.

It is believed that the results indicate that the ultrasonic nebulising process results in a most effective relative enrichment of leucine concentration at the particle surface. The surface enrichment is dependent upon the rate of leucine transport to the surface, the size of the particle, and its precipitation rate, during the drying process. This precipitation rate is related to the slow drying of the particles in this process. The resulting effect is that the particle surface is dominated by the hydrophobic aspects of the leucine. This presents a relatively low surface energy of the powder despite its small particle size and high surface area. It therefore appears that the addition of a force control agent is having a superior influence to adhesivity and cohesivity and hence the device retention and dispersion.

The inclusion of leucine appears to provide significant improvements to the aerosolisation of clomipramine hydrochloride, and should make this drug suitable for use in a high-dose passive or active device.

### EXAMPLE

### Preparation of pMDI Formulation

A further composition according to the present invention may be prepared as follows. 12.0 g micronised antidepressant, such as clomipramine, and 4.0 g lecithin S PC-3 (Lipoid GMBH) are weighed into a beaker. The powder is transferred to the Hosokawa AMS-MINI MechanoFusion system via a funnel attached to the largest port in the lid with the equipment running at 3.5%. The port is sealed and the cooling water switched on. The equipment is run at 20% for 5 minutes followed by 50% for 10 minutes. The equipment is switched off, dismantled and the resulting formulation recovered mechanically.

Preparation of Cans:

0.027 g powder is weighed into the can, a 50 μl valve is crimped to the can and 12.2 g HFA 134a is back filled into the can.

### EXAMPLE

### Preparation of MechanoFused Formulation for Use in Passive Device

A further composition according to the present invention may be prepared as follows. 20 g of a mix comprising 20% micronised antidepressant, such as clomipramine, 78% Sorbolac 400 lactose and 2% magnesium stearate are weighed into the Hosokawa AMS-MINI MechanoFusion system via a funnel attached to the largest port in the lid with the equipment running at 3.5%. The port is sealed and the cooling water switched on. The equipment is run at 20% for 5 minutes followed by 80% for 10 minutes. The equipment is switched off, dismantled and the resulting formulation recovered mechanically.

1. A composition for treating premature ejaculation by pulmonary inhalation, said composition comprising an antidepressant.

2. A composition as claimed in claim 1, wherein the antidepressant is a tricyclic antidepressant.

3. A composition as claimed in claim 1, wherein the composition comprises two or more antidepressants.

4. A composition as claimed in claim 1, wherein the composition comprises a further therapeutic agent, which is not an antidepressant.

5. A composition as claimed in claim 4, wherein the further therapeutic agent is also effective in treating PE.

6. A composition as claimed in claim 4, wherein the further therapeutic agent is a benzodiazepine.

7. A composition as claimed in claim 1, wherein the administration of the composition by pulmonary inhalation is not accompanied with the adverse side effects usually associated with the administration of the antidepressant.

8. A composition as claimed in claim 1, wherein the composition provides a dose of antidepressant of less than about 25 mg.

9. A composition as claimed in claim 1, wherein the composition provides an onset of the therapeutic effect within no more than 30 minutes following pulmonary administration.

10. A composition as claimed in claim 1, wherein the composition is a dry powder composition.

11. A composition as claimed in claim 10, wherein the composition comprises particles of antidepressant having a mass median aerodynamic diameter of about 10 μm or less.

12. A composition as claimed in claim 11, wherein the mass median aerodynamic diameter is about 5 μm or less.

13. A composition as claimed in claim 10 wherein at least 90% of the antidepressant has a particle size of about 10 μm or less.

14. A composition as claimed in claim 13, wherein at least 90% of the antidepressant has a particle size of about 5 μm or less.

15. A composition as claimed in claim 10, wherein the composition further comprises an additive material.

16. A composition as claimed in claim 15, wherein the additive material is provided in an amount from about 0.15% to about 5% of the composition, by weight.

17. A composition as claimed in claim 15, wherein the additive material is selected from the group consisting of leucine, magnesium stearate, lecithin, and sodium stearyl fumarate.

18. A composition as claimed in claim 10, wherein the composition further comprises an excipient material.

19. A composition as claimed in claim 18, wherein the excipient material is in the form of carrier particles having an average particle size of about 40 to about 70 μm.

20. A composition as claimed in claim 1, wherein the composition comprises a solution pMDI formulation including a propellant, a solvent and water.

21. A composition as claimed in claim 1, wherein the composition is a suspension pMDI formulation including a propellant.

22. A composition as claimed in claim 20, wherein the propellant is selected from the group consisting of: HFA134a HFA227 and a combination thereof.

23. A method of treating premature ejaculation, the method comprising administering to a subject in need of such treatment a composition as claimed in claim 1.

24. A method as claimed in claim 23, wherein the method does not cause the adverse side effects normally associated with the administration of the antidepressant.

25-26. (canceled)

27. A dry powder inhaler device comprising a composition as claimed in claim 1.

28. A dry powder inhaler device as claimed in claim 27, wherein the inhaler is an active inhaler.

29. A dry powder inhaler device as claimed in claim 27, wherein the inhaler is a breath actuated inhaler device.

30. The device of claim 27 comprising a blister, wherein the blister contains the composition.

31. The method of claim 23 wherein adverse side effects, if any, provoked by the administration of the composition by inhalation are such that they would easily be tolerated by an average recipient.

32. A composition as claimed in claim 1, wherein the composition provides a dose of antidepressant of less than about 15 mg.

33. A composition as claimed in claim 1, wherein the composition provides a dose of antidepressant of less than about 5 mg.

34. A composition as claimed in claim 1, wherein the composition provides an onset of the therapeutic effect within no more than 20 minutes following pulmonary administration.

35. A composition as claimed in claim 1, wherein the composition provides an onset of the therapeutic effect within no more than 10 minutes following pulmonary administration.

36. A composition as claimed in claim 1, wherein the composition provides an onset of the therapeutic effect within no more than 5 minutes following pulmonary administration.

37. A composition as claimed in claim 1, wherein the composition provides an onset of the therapeutic effect within no more than 1 minute following pulmonary administration.
